(19) United States
(12) Patent Application Publication
(10) Pub. No.: US 2009/0182044 A1
(43) Pub. Date: Jul. 16, 2009

(54) NITRATE AMINO ACID CHELATES

(76) Inventors: H. DeWayne Ashmed, Fruit Heights, UT (US); Charlie Thompson, Morgan, UT (US)

Correspondence Address: THORPE NORTH & WESTERN, LLP., P.O. Box 1219, SANDY, UT, 84091-1219, US

(21) Appl. No.: 12/351,677

(22) Filed: Jan. 9, 2009

Related U.S. Application Data
(60) Provisional application No. 61/010,721, filed on Jan. 11, 2008.

(51) Int. Cl. A61K 31/315; C07C 203/04; C12N 5/00; A61K 31/21; A01N 33/16; C07F 9/00; C07F 13/00; C07F 11/00; C07F 1/00; C07F 3/02; C07F 15/02; C07F 7/02; C07F 1/08; C07F 3/04; C07F 3/06; C07F 15/04; C07F 15/06; A61K 31/30; A61K 31/295; A61K 31/28; A01N 55/02; A61P 3/00

(52) U.S. Cl. 514/494; 558/483; 435/375; 435/377; 514/509; 504/326; 556/42; 556/45; 556/57; 556/110; 556/118; 556/138; 556/400; 514/499; 514/501; 514/502; 514/492; 504/190

(57) ABSTRACT

The present invention is directed to methods and compositions which include nitrate amino acid chelates that can increase the metabolic activity or metal concentration in animals and that can increase metabolic activity and nitrogen content in plants. In one embodiment, a nitrate-complexed amino acid composition can comprise a metal, an amino acid ligand, and a nitrate, wherein the amino acid ligand is chelated to the metal forming an amino acid chelate and the nitrate is complexed to the amino acid chelate. In another embodiment, a nitrate-chelated amino acid composition can comprise a metal, an amino acid ligand, and a nitrate, wherein the amino acid ligand and the nitrate are chelated to the metal forming a nitrate-chelated amino acid chelate.

The present application claims the benefit of U.S. Provisional Patent Application Ser. No. 61/010,721, filed on Jan. 11, 2008, which is hereby incorporated by reference in its entirety.

### BACKGROUND

Amino acid chelates are generally produced by the reaction between α-amino acids and metal ions having a valence of two or more to form a ring structure. In such a reaction, the positive electrical charge of the metal ion can be neutralized by the electrons available through the carboxylate or free amino groups of the α-amino acid.

Traditionally, the term “chelate” has been loosely defined as a combination of a metallic ion bonded to one or more ligands to form a heterocyclic ring structure. Under this definition, chelate formation through neutralization of the positive charge(s) of the metal ion may be through the formation of ionic, covalent or coordinate covalent bonding. An alternative and more modern definition of the term “chelate” requires that the metal ion be bonded to the ligand solely by coordinate covalent bonds forming a heterocyclic ring. In either case, both are definitions that describe a metal ion and a ligand forming a heterocyclic ring.

Chelation can be confirmed and differentiated from mixtures of components or more ionic complexes by infrared spectra through comparison of the stretching of bonds or shifting of absorption caused by bond formation. As applied in the field field of mineral nutrition, there are certain “chelated” products that are commercially utilized. The first is referred to as a “metal proteinate.” The American Association of Feed Control officials (AAFCO) has defined a “metal proteinate” as the product resulting from the chelation of a soluble salt with amino acids and/or partially hydrolyzed protein. Such products are referred to as the specific metal proteinate, e.g., copper proteinate, zinc proteinate, etc. Sometimes, metal proteinates are erroneously referred to as “amino acid” chelates.

The second product, referred to as an “amino acid chelate,” when properly formed, is a stable product having one or more five-membered rings formed by a reaction between the amino acid and the metal. The American Association of Feed Control Officials (AAFCO) has also issued a definition for metal amino acid chelates. It is officially defined as the product resulting from the reaction of a metal ion from a soluble metal salt with amino acids having a mole ratio of one mole of metal to one to three (preferably two) moles of amino acids to form coordinate covalent bonds. The average weight of the hydrolyzed amino acids must be approximately 150 and the resulting molecular weight of the chelate must not exceed 800. The products are identified by the specific metal forming the chelate, e.g., iron amino acid chelate, copper amino acid chelate, etc.

In further detail with respect to amino acid chelates, the carboxyl oxygen and the α-amino group of the amino acid each bond with the metal ion. Such a five-membered ring is defined by the metal atom, the carboxyl oxygen, the carbonyl carbon, the α-carbon and the α-amino nitrogen. The actual structure will depend upon the ligand to metal mole ratio and whether the carboxyl oxygen forms a coordinate covalent bond or an ionic bond with the metal ion. Generally, the ligand to metal molar ratio is at least 1:1 and is preferably 2:1 or 3:1. However, in certain instances, the ratio may be 4:1. Most typically, an amino acid chelate with a divalent metal can be represented at a ligand to metal molar ratio of 2:1 according to Formula 1 as follows:

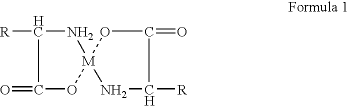

In the above formula, the dashed lines represent coordinate covalent bonds, covalent bonds, or ionic bonds. Further, when R is H, the amino acid is glycine, which is the simplest of the α-amino acids. However, R could be representative of any other side chain that, when taken in combination with the rest of the ligand structure(s), results in any of the other twenty or so naturally occurring amino acids used in protein synthesis. All of the amino acids have the same configuration for the positioning of the carboxyl oxygen and the α-amino nitrogen with respect to the metal ion. In other words, the chelate ring is defined by the same atoms in each instance, even though the R side chain group may vary.

With respect to both amino acid chelates and proteinates, the reason a metal atom can accept bonds over and above the oxidation state of the metal is due to the nature of chelation. For example, at the α-amino group of an amino acid, the nitrogen contributes to both electrons used in the bonding. These electrons fill available spaces in the d-orbitals of the metal ion forming a coordinate covalent bond. Thus, a metal ion with a normal valency of +2 can be bonded by four bonds when fully chelated. In this state, the chelate is completely satisfied by the bonding electrons and the charge on the metal atom (as well as on the overall molecule) is zero. As stated previously, it is possible that the metal ion can be bonded to the carboxyl oxygen by either coordinate covalent bonds or ionic bonds. However, the metal ion is preferably bonded to the α-amino group by coordinate covalent bonds only.

The structure, chemistry, bioavailability, and various applications of amino acid chelates are well documented in the literature, e.g. Ashmead et al., Chelated Mineral Nutrition, (1982), Chas. C. Thomas Publishers, Springfield, Ill.; Ashmead et al., Intestinal Absorption of Metal Ions, (1985), Chas. C. Thomas Publishers, Springfield, Ill.; U.S. Pat. Nos. 4,020,158; 4,167,564; 4,216,143; 4,216,144; 4,599,152; 4,725,427; 4,774,089; 4,830,716; 4,863,898; 5,292,538; 5,292,729; 5,516,925; 5,596,016; 5,882,685; 6,159,530; 6,166,071; 6,207,204; 6,294,207; and 6,614,553; each of which are incorporated herein by reference.

One advantage of amino acid chelates in the field of mineral nutrition is attributed to the fact that these chelates are readily absorbed from the gut and into mucosal cells by means of active transport. In other words, the minerals can be absorbed along with the amino acids as a single unit utilizing the amino acid(s) as a carrier molecule. Therefore, the problems associated with the competition of ions for active sites and the suppression of specific nutritive mineral elements by others can be avoided.

As such, metal amino acid chelates have been used as a dietary supplement for a variety of nutritional metals and amino acids. Even though chelation generally offers better mineral absorbability, absorption is a complex biological function influenced by many variables. As such, methods and complexes with improved absorption characteristics and that provide increased health benefits continue to be sought through ongoing research and development efforts.

### SUMMARY

Briefly, and in general terms, the invention is directed to methods and compositions that are formulated such that nitrate amino acid chelates can increase the metabolic activity and metal tissue concentration in an animal. In one one embodiment, a nitrate-complexed amino acid chelate composition can comprise a metal, an amino acid ligand, and a nitrate, such that the amino acid ligand is chelated to the metal forming an amino acid chelate and the nitrate is complexed to the amino acid chelate. The composition can further include a second amino acid chelate or a nitrate salt.

In another embodiment, a nitrate-chelated amino acid chelate composition can comprise a metal, an amino acid ligand, and a nitrate, such that the amino acid ligand and the nitrate are chelated to the metal forming a nitrate-chelated amino acid chelate.

Additionally, a method of increasing a metabolic activity in an animal tissue can comprise administering a nitrate-complexed amino acid chelate composition or nitrate-chelated amino acid chelate composition as previously described to an animal in an amount sufficient to i) raise the metal concentration within the tissue, ii) retain metal content in the tissue for a greater period of time compared to when the metal is delivered as a non-nitrate-containing compound, and/or iii) enhance metabolic activity of the tissue.

In one embodiment, a method of increasing a metabolic activity in a plant can comprise administering a nitrate amino acid chelate composition including a metal, an amino acid ligand, and a nitrate; where the amino acid ligand is chelated to the metal forming an amino acid chelate and the nitrate is chelated or complexed to the amino acid chelate, and where the composition is administered to the plant in an amount sufficient to raise the nitrogen concentration within the plant and enhance metabolic activity of the plant.

Other embodiments will also be described herein which illustrate, by way of example, features of the present invention.

### DETAILED DESCRIPTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It is noted that, as used in this specification and the appended claims, the singular forms “a,” “an,” and, “the” include plural referents unless the context clearly dictates otherwise. Thus, for example, reference to “a chelate” can include one or more of such chelates, reference to “an amount of nitrates” can include reference to one or more amounts of nitrates, and reference to “the amino acid” can include reference to one or more amino acids.

As used herein, the term “naturally occurring amino acid” or “traditional amino acid” shall mean amino acids that are known to be used for forming the basic constituents of proteins, including alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof.

As used herein, the term “nitrate-complexed amino acid chelate” refers to an amino acid chelate with at least one nitrate having an ionic, covalent, coordinate, or coordinate-covalent bond with the amino acid chelate. For example, the following formula represents a nitrate-complexed amino acid chelate in accordance with one embodiment of the present invention:

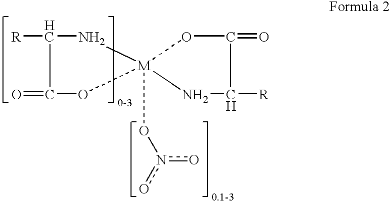

In the above formula, the dashed lines represent coordinate covalent bonds, covalent bonds, ionic bonds, or resonance bonds between nitrogen and oxygen in the case of the nitrate ion. Further, when R is H, the amino acid is glycine, which is the simplest of the α-amino acids. However, R could be representative of any other side chain that, when taken in combination with the rest of the ligand structure(s), results in any of the other twenty or so naturally occurring amino acids used in protein synthesis. All of the amino acids have the same configuration for the positioning of the carboxyl oxygen and the α-amino nitrogen with respect to the metal ion. In other words, the chelate ring is defined by the same atoms in each instance, even though the R side chain group may vary. M represents any divalent or trivalent metal as defined herein.

As used herein, the term “nitrate-chelated amino acid chelate” refers to an amino acid chelate having at least one nitrate chelated to the metal through the oxylate anions of the nitrate forming a 4-membered ring. For example, the following formula represents a nitrate-chelated amino acid chelate in accordance with one embodiment of the present invention:

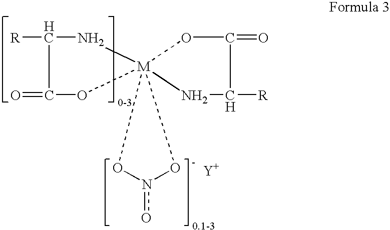

In the above formula, the dashed lines represent coordinate covalent bonds, covalent bonds, or ionic bonds. Further, when R is H, the amino acid is glycine, which is the simplest of the α-amino acids. However, R could be representative of any other side chain that, when taken in combination with the rest of the ligand structure(s), results in any of the other twenty or so naturally occurring amino acids used in protein synthesis. All of the amino acids have the same configuration for the positioning of the carboxyl oxygen and the α-amino nitrogen with respect to the metal ion. In other words, the chelate ring is defined by the same atoms in each instance, even though the R side chain group may vary. M represents any divalent or trivalent metal as defined herein. Y represents any monovalent or divalent counterion appropriate for use with the nitrate anion. However, such a counterion may be absent depending on the nitrate sources used. For example, if ferric nitrate Fe(NO₃)₃ was used as a source of the metal ion, only a proper amount of an amino acid would be needed to make a compound that was a nitrate chelate/complex, but it would be free of the Y⁺ counterions. However, if potassium nitrate KNO₃ and Zinc carbonate were used, the counterion would be a monovalent potassium ion (Y⁺) or if magnesium nitrate Mg(NO₃)₂ and calcium hydroxide with aspartic acid were used, the counterion would be divalent Mg⁺². It is noted that the term, “nitrate chelate/complexed” or the like refers to a composition having at least a portion of the like refers to a composition having at least a portion of the nitrates chelated to the metals, i.e., in the form of a “nitrate-chelated amino acid chelates” as defined herein, but may also contain “nitrate-complexed amino acid chelates” as defined herein. In other words, at least a portion of the nitrates are chelated to the metal, thus, being a “nitrate-chelated amino acid chelates.”

As used herein, the term “amino acid chelate” refers to both the traditional definitions and the more modern definition of chelate as cited previously. Specifically, with respect to chelates that utilize traditional amino acid ligands, i.e., those used in forming proteins, chelate is meant to include metal ions bonded to proteinaceous ligands forming heterocyclic rings. Between the carboxyl oxygen and the metal, the bond can covalent or ionic, but is preferably coordinate covalent. Additionally, at the α-amino group, the bond is typically a coordinate covalent bond. Proteinates of naturally occurring amino acids are included in this definition.

As used herein, the term “metal” refers to nutritionally relevant metals including divalent and trivalent metals that can be used as part of a nutritional supplement, are known to be beneficial to humans, and are substantially non-toxic when administered in traditional amounts, as is known in the art. Examples of such metals include copper, zinc, manganese, iron, chromium, calcium, potassium, sodium, magnesium, cobalt, nickel, molybdenum, vanadium, strontium, selenium, and the like. This term also includes nutritional semi-metals including, but not limited to, silicon.

As used herein, the term “proteinate” when referring to a metal proteinate is meant to include compounds where the metal is chelated or complexed to hydrolyzed or partially hydrolyzed protein forming a heterocyclic ring. Coordinate covalent bonds, covalent bonds, and/or ionic bonds may be present between the metal and the proteinaceous ligand of the chelate or chelate/complex structure. As used herein, proteinates are included when referring to amino acid chelates. However, when a proteinate is specifically mentioned, it does not include all types of amino acid chelates, as it only includes those with hydrolyzed or partially hydrolyzed protein.

As used herein, the term “amino acid chelate” and “metal amino acid chelate” are used interchangeable, as by definition, a chelate requires the presence of a metal.

As used herein, the term “nitrate amino acid chelate” refers to both nitrate-complexed amino acid chelates and nitrate-chelated amino acid chelates, as defined herein.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 micron to about 5 microns” should be interpreted to include not only the explicitly recited values of about 1 micron to about 5 microns, but also include individual values and sub-ranges within the ranges within the indicated range. Thus, included in this numerical range are individual values such as 2, 3.5, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

With these definitions in mind, nitrate amino acid chelates can increase metabolic activity in an animal as well as mineral adsorption in an animal tissue. Additionally, these compounds could also be used for plants. Generally, chelation has been shown to increase the absorbability of minerals since they are readily absorbed from the gut and into mucosal cells by means of active transport. In other words, the minerals are often absorbed along with the amino acids as a single unit, thereby utilizing the amino acids as carrier molecules. This being stated, it has been found that nitrate amino acid chelates have an unexpected effect on the metabolic activity of various animals. Generally, the nitrate amino acid chelates can increase the mineral concentration in the animal tissue and retain the metal in the tissue for a longer period of time. For example, in mammals, e.g., cows, sows, poultry, etc., metabolic activity such as milk production, weight gain, fertility, feed conversion, etc. can be increased by such administration more so than by delivering metal compounds without nitrate amino acid chelates. Additionally, such increased metabolic activity can provide an increased quantity and quality of associated products, such as, but not limited to, milk products and/or meat. Furthermore, the increased metabolic activity can reduce morbidity and mortality.

In one embodiment, a nitrate-complexed amino acid chelate composition can comprise a metal, an amino acid ligand, and a nitrate, such that the amino acid ligand is chelated to the metal forming an amino acid chelate and the nitrate is complexed to the amino acid chelate.

As defined in Formula 2 above, a nitrate-complexed amino acid chelate can be represented by the following formula:

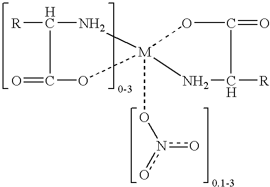

where the dashed lines represent coordinate covalent bonds, covalent bonds, or ionic bonds. Further, when R is H, the amino acid is glycine, which is the simplest of the α-amino acids. However, R can be representative of any other side chain that, when taken in combination with the rest of the ligand structure(s), results in any of the other twenty or so naturally occurring amino acids used in protein synthesis, e.g., alanine, arginine, asparagine, aspartic acid, cysteine, cystine, glutamine, glutamic acid, histidine, hydroxyproline, isoleucine, leucine, lysine, methionine, ornithine, phenylalanine, proline, serine, threonine, tryptophan, tyrosine, valine, and combinations thereof. All of the amino acids have the same configuration for the positioning of the carboxyl oxygen and the α-amino nitrogen with respect to the metal ion. In other words, the chelate ring is defined by the same atoms in each instance, even though the R side chain group may vary. M represents any divalent or trivalent nutritionally relevant metal as defined herein, e.g., copper, zinc, manganese, iron, chromium, calcium, potassium, sodium, magnesium, cobalt, nickel, molybdenum, vanadium, strontium, and selenium, as well as nutritional semi-metals including, but not limited to, silicon. Y represents any appropriate monovalent or divalent counterion (cation) for the nitrate anion. For example, Y can be sodium, lithium; potassium; ammonium; mono-, di-, and trimethylammonium, quaternary amines; or the like. As shown, the composition has an amino acid ligand to metal ratio of about 1:1 to about 3:1 and has a nitrate to metal ratio of about 0.1:1 to about 1:3. As such, the composition may contain amino acid chelates that are not complexed to a nitrate; however, the compound, as a whole, contains about 0.1 to about 3 nitrate per amino acid chelate. Additionally, the amount of ligands present is dependent on the valency of the metal used. For example, it is possible for divalent cations to coordinate with up to 8 ligands. As such, a number of combinations can be envisioned by the above formula. Specifically, in one embodiment, the nitrate-complexed amino acid chelate can have 2 amino acid ligands and 1 nitrate. In another embodiment, the nitrate-complexed amino acid chelate can have 3 amino acid ligands and 1 nitrate. In another embodiment, the nitrate-complexed amino acid chelate can have 1 amino acid ligand and 1 nitrate. In another embodiment, a nitrate-complexed amino acid chelate having a divalent cation can have 2 amino acid ligands with 2 nitrates.

The composition can further include a second amino acid chelate or a nitrate salt. In one embodiment, the second amino acid chelate or nitrate salt can be admixed with the nitrate-complexed amino acid chelate. Additionally, the second amino acid chelate or nitrate salt can be present in the composition in a ratio of about 1:10 to about 10:1. The second amino acid chelate can contain a second metal and a second amino acid ligand, such that the second amino acid chelate is different than the amino acid chelate complexed to the nitrate. In one embodiment, the metals are different and the amino acid ligands are the same. In another embodiment, the metals are the same while the amino acids are different. In still another embodiment, both the metals and the ligands are different. It is noted that in one embodiment, the second amino acid chelate can be a second nitrate-complexed amino acid chelate. Additionally, in one embodiment, the second amino second amino acid chelate can be a nitrate-chelated amino acid chelate.

In an alternative embodiment, a nitrate-chelated amino acid chelate can comprise a metal, an amino acid ligand, and a nitrate, such that the amino acid ligand and the nitrate are chelated to the metal forming a nitrate-chelated amino acid chelate. As previously set forth in Formula 3 above, the nitrate-chelated amino acid chelate can be represented by the following formula:

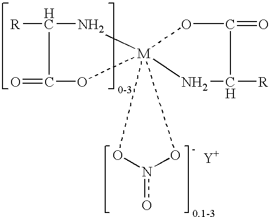

where the dashed lines represent coordinate covalent bonds, covalent bonds, or ionic bonds. Further, when R is H, the amino acid is glycine, which is the simplest of the α-amino acids. However, R can be representative of any other side chain that, when taken in combination with the rest of the ligand structure(s), results in any of the other twenty or so naturally occurring amino acids used in protein synthesis, e.g., alanine, arginine, asparagine, aspartic acid, cysteine, cystine, glutamine, glutamic acid, histidine, hydroxyproline, isoleucine, leucine, lysine, methionine, ornithine, phenylalanine, proline, serine, threonine, tryptophan, tyrosine, valine, and combinations thereof. All of the amino acids have the same configuration for the positioning of the carboxyl oxygen and the α-amino nitrogen with respect to the metal ion. In other words, the chelate ring is defined by the same atoms in each instance, even though the R side chain group may vary. Additionally, the nitrate chelates the metal by forming a 4-membered ring through the oxylate anions of the nitrate. M represents any divalent or trivalent nutritionally relevant metal as defined herein, e.g., copper, zinc, manganese, iron, chromium, calcium, potassium, sodium, magnesium, cobalt, nickel, molybdenum, vanadium, strontium, and selenium, as well as nutritional semi-metals including, but not limited to, silicon. Y represents any appropriate monovalent or divalent counterion (cation) for the nitrate anion. For example, Y can be sodium; lithium; potassium; ammonium; magnesium; mono-, di-, and trimethlyammonium; quaternary amines; or the like. As shown, the composition has an amino acid ligand to metal ratio of about 1:1 to about 3:1 and has a nitrate to metal ratio of about 0.1:1 to about 3:1. As such, the composition may contain amino acid chelates that are not chelated to a nitrate; however, the compound, as a whole, contains about 0.1 to about 3 nitrates per amino acid chelate. Additionally, the amount of ligands present is dependent on the valency of the metal used. As such, a number of combinations can be envisioned by the above formula. Specifically, in one embodiment, the nitrate-chelated amino acid chelate can have 2 amino acid ligands and 1 nitrate. In another embodiment, the nitrate-chelated amino acid chelate can have 3 amino acid ligands and 1 nitrate. In another embodiment, the nitrate-chelated amino acid chelate can have 1 amino acid ligand and 1 nitrate. In yet another embodiment, the nitrate-chelated amino acid chelate can have 2 amino acid ligands and 2 nitrates. In still yet another embodiment, the nitrate-chelated amino acid chelate can have 1 amino acid ligand and 3 nitrates. In still yet another embodiment, the nitrate-chelated amino acid chelate can have 1 amino acid ligand and 2 nitrates.

As discussed above, the composition can further include a second amino acid chelate or a nitrate salt. In one embodiment, the second amino acid chelate or nitrate salt can be admixed with the nitrate-chelated amino acid chelate. Additionally, the second amino acid chelate or nitrate salt can be present in the composition in a ratio of about 1:10 to about 10:1. The second amino acid chelate can contain a second metal and a second amino acid ligand, such that the second amino acid chelate is different than the amino acid chelate chelated to the nitrate. In one embodiment, the metals are different and the amino acid ligands are the same. In another embodiment, the metals are the same while the amino acids are different. In still another embodiment, both the metals and the ligands are different. It is noted that in one embodiment, the second amino acid chelate can be a second nitrate-chelated amino acid chelate. Additionally, in one embodiment, the second amino acid chelate can be a nitrate-complexed amino acid chelate.

Additionally, a method of increasing a metabolic activity in an animal tissue can comprise administering a nitrate amino acid chelate, e.g., a nitrate-complexed amino acid chelate composition and/or nitrate-chelated amino acid chelate composition, to an animal in an amount sufficient to i) raise the metal concentration within the tissue, ii) retain metal content in the tissue for a greater period of time compared to when the metal is delivered as a non-nitrate-containing compound, and/or iii) enhance metabolic activity of the tissue.

In one embodiment, the methods can further comprise coadministering a second amino acid chelate or nitrate salt, including the types and ratios of second amino acid chelates and nitrate salts previously described and further described herein. As such, the methods described herein contemplate the use of different amino acid chelates including additional nitrate amino acid chelates.

Additionally, a method of increasing a metabolic activity in a plant can comprise administering a nitrate amino acid chelate composition including a metal, an amino acid ligand, and a nitrate; where the amino acid ligand is chelated to the metal forming an amino acid chelate and the nitrate is chelated or complexed to the amino acid chelate, and where the composition is administered to the plant in an amount sufficient to raise the nitrogen concentration within the plant and enhance metabolic activity of the plant.

Nitrate (N), phosphate (P), and potassium (K) are all essential for plant growth, so these compounds could be used with metal nitrates or as a source of metal nitrates blended with potassium, phosphate, and nitrate for optimum plant nutrition. Common (NPK) sources and fertilizer materials, such as ammonium nitrate, ammonium phosphate, monoammonium phosphate, ammonium nitrate-sulfate, ammonium phosphate sulfate, ammonium phosphate nitrate, ammonium polysulfide, diammonium phosphate, ammonium sulfate, potassium nitrate, potassium phosphate, potassium chloride, potassium sulfate, potassium thiosulfate, potassium magnesium sulfate, single superphosphate, triple superphosphate, phosphoric acid, superphosphoric acid, ammonium thiosulfate, anhydrous ammonia, aqua ammonia, calcium ammonium nitrate solution, calcium nitrate, calcium cyanamide, sodium nitrate, urea, methylene ureas, urea ammonium nitrate solution, and mixtures thereof, can be mixed with the nitrate amino acid chelates described herein to provide very effective high nitrate plant fertilizers. The present nitrate amino acid chelates can also be mixed as multi-mineral dry blended or foliar fertilizers. Such compositions can contain optimized nitrogen/nitrate to mineral ratio for optimum plant growth or other metabolic activity and can be very effective in simultaneously supplying minerals and nitrate to plants. In one embodiment, the metabolic activity can be enhanced growth, enhanced fruit production, reduced morbidity, or enhanced fruit size. The administration can be by foliar plant fertilizer, solid plant fertilizer, liquid plant fertilizer, or combinations thereof.

Generally, the amino acid chelates contemplated for use in the compositions and methods of the present invention can include amino acid ligands such as, but not limited to, alanine, arginine, asparagine, aspartic acid, cysteine, cystine, glutamine, glutamic acid, glycine, histidine, hydroxyproline, isoleucine, leucine, isoleucine, leucine, lysine, methionine, ornithine, phenylalanine, proline, serine, threonine, tryptophan, tyrosine, and valine, including dipeptides, tripeptides, and tetrapeptides thereof.

Additionally, the metals contemplated for use in the compositions and methods of the present invention can be generally nutritionally relevant metals, as defined previously. Specific examples include, but are not limited to, copper, zinc, manganese, iron, chromium, calcium, potassium, sodium, magnesium, cobalt, nickel, molybdenum, vanadium, strontium, and selenium, as well as semi-metals, such as silicon. It is noted that certain metals may perform better for certain targeted metabolic activity. For example, if the desire is to enhance general growth, metals such as zinc, iron, or calcium may be preferable for use in the amino acid chelate and/or the amino acid complex (which may optionally also be a chelate). If the desire is to enhance milk production, metals such as manganese, zinc, calcium, or copper may be preferable for use in the amino acid chelate and/or the amino acid complex (which may also optionally also be a chelate). If the desire is to enhance reproduction, metals such as zinc or manganese may be used in the amino acid chelate and/or the amino acid complex (which may also optionally also be a chelate). Other metabolic activities and metal choices may be determined by one skilled in the art. If the desire is to reduce infant mortality, iron may be preferably for use in the amino acid chelate and/or the amino acid complex (which may also optionally also be a chelate). Generally, the methods and compositions can be formulated for any animal, e.g., humans, mammals, fowl, fish, crustacean, etc, or plant.

An amino acid chelate composition can include numerous combinations of metals to ligands in the form of chelates and other compounds and complexes. Such arrangements are contemplated by the present invention and may be manufactured through generally known preparative complex and/or chelation methods. It is not the purpose of the present invention to describe how to prepare amino acid chelates that can be used with the present invention. Suitable methods for preparing such amino acid chelates can include those described in U.S. Pat. Nos. 4,830,716 and/or 5,516,925, to name a few. However, combinations of such chelates as part of a composition for increasing metabolic activity or increasing and retaining metal content in a tissue are included as an embodiment of the present invention.

In the compositions and methods described herein, nitrate salts include, without limitation, group 1 element nitrates; group 2 element nitrates; transitional metal nitrates; amino acid nitrates; quaternary amine nitrates; mono-, di-, and trimethylaminenitrates; including HNO₃, LiNO₃, Be(NO₃)₂, NaNO₃, Mg(NO₃)₂, KNO₃, Ca(NO₃)₂, Cr(NO₃)₃, Mn(NO₃)₂, Fe(NO₃)₃, Co(NO₃)₂, Ni(NO₃)₂, Cu(NO₃)₂, Zn(NO₃)₂, Sr(NO₃)₂, etc.

In each of the above-described embodiments, the compositions and methods of the present invention can provide a nitrogen content to an animal from about 5 wt % to about 60 wt %, based on the composition as a whole. Additionally, the compositions and methods of the present invention can provide metal content to an animal from about 5 wt % to about 45 wt %. Also as previously mentioned, the metabolic activity can enhance milk production, weight gain, enhanced growth, enhanced fertility, reduced morbidity, reduced tissue fat, or enhanced feed conversion. The compositions can be formulated for parenteral delivery. The compositions for administration can have formulations including oral, injection, powder, tablet, capsule, gel, liquid, or paste. In one embodiment, the step of administering can be oral administration.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

### EXAMPLES

The following provides examples of high nitrogen amino acid compositions in accordance with the compositions and methods previously disclosed. Additionally, some of the examples include studies performed showing the effects of high nitrogen metal amino acid chelates on animals in accordance with embodiments of the present invention.

### Example 1

### Nitrate Complexed Iron Arginine Chelate

To about 1200 ml of deionized water containing 71 grams nitric acid, 386 grams of arginine is added to form a clear solution. To this solution of nitric acid and arginine, 62 grams of elemental iron is slowly added. The solution is heated at about 50° C. for 8 hours, or until substantially all the iron is observed to go into solution. The product is cooled, filtered, and dried yielding a nitrate-complexed ferrous bisarginate amino acid chelate.

### Example 2

### Nitrate Complexed Magnesium Arginine Chelate

A nitrate-complexed amino acid chelate magnesium nitrate composition is obtained by dry blending 470.6 grams of the nitrate-complexed ferrous bisarginate amino acid chelate with 148.31 grams of magnesium nitrate to provide a homogenous nitrate amino acid composition with a molar ratio of nitrate-complexed amino acid chelate to magnesium nitrate ratio of about 1:1.

### Example 3

### Nitrate Complexed Iron Glycine Chelate

A nitrate-complexed amino acid chelate potassium nitrate composition is obtained by dry blending 275.7 grams of the nitrate-complexed ferrous bisglycinate amino acid chelate with 101.1 grams of potassium nitrate to provide a homogenous nitrate amino acid composition with a molar ratio of nitrate-complexed amino acid chelate to potassium nitrate ratio of about 1:1.

### Example 4

### Nitrate Complexed Iron Arginine Chelate

To about 1200 ml of deionized water containing 71 grams nitric acid, 386 grams of arginine is added to form a clear solution. To this solution of nitric acid and arginine, 62 grams of elemental iron is slowly added. The solution is heated at about 50° C. for 8 hours, or until substantially all the iron is observed to go into solution. The product is cooled, and dried yielding a nitrate-complexed ferrous bisarginate amino acid chelate.

### Example 5

### Nitrate Complexed Manganese Glycine Chelate

To about 1200 ml of deionized water is added 295 grams of manganese nitrate. To this solution, 245 grams of glycine is slowly added. The solution is heated at about 50° C. for 8 hours, or until substantially all the manganese is observed to go into solution. The product is cooled, and dried yielding a nitrate-complexed manganese bisglycinate amino acid chelate.

### Example 6

### Nitrate Complexed Manganese Glycine Chelate

To about 1200 ml of deionized water containing 122 grams nitric acid, 285 grams of glycine is added. To this solution of nitric acid and glycine, 62 grams of elemental manganese is slowly added. The solution is heated at about 50° C. for 8 hours, or until substantially all the manganese is observed to go into solution. The product is cooled, and dried yielding a nitrate-complexed manganese bisglycinate amino acid chelate

### Example 7

### Nitrate Complexed Zinc Arginine Chelate

To about 1200 ml of deionized water containing 125 grams nitric acid, 345 grams of arginine is added to form a clear solution. To this solution of nitric acid and arginine, 80 grams of elemental zinc oxide is slowly added. The solution is heated at about 50° C. for 8 hours. The product is cooled, and dried yielding a nitrate-complexed zinc bisarginate amino acid chelate complex.

### Example 8

### Nitrate Chelate/Complexed Zinc Arginine Chelate

To about 1200 ml of deionized water is added 180 grams of zinc nitrate. To this solution of zinc nitrate, 330 grams of arginine is slowly added. The solution is heated at about 50° C. for 4 hours. The product is cooled, and dried yielding a nitrate chelated amino acid chelate, which can more specifically be in the form of a nitrate chelate/complexed zinc bisarginate amino acid chelate complex.

### Example 9

### Nitrate Complexed Calcium Asparagine Chelate

To about 1200 ml of deionized water containing 94 grams nitric acid, 390 grams of asparagine is added to form a clear solution. To this solution of nitric acid and asparagine, 105 grams of calcium oxide is slowly added. The solution is heated at about 50° C. for 8 hours. The product is cooled, and dried yielding a nitrate-complexed calcium bisasparagine amino acid chelate complex.

### Example 10

### Nitrate Complexed Copper Lysine Chelate

To about 1200 ml of deionized water containing 82 grams nitric acid, is added 360 grams of lysine to form a clear solution. To this solution of nitric acid and lysine, 120 grams of copper hydroxide is slowly added. The solution is heated at about 50° C. for 4 hours, cooled, and dried yielding a nitrate-complexed cupric bislysinate amino acid chelate.

### Example 11

### Nitrate Complexed Copper Isoleucine Chelate

To about 1200 ml of deionized water is added 147 grams of copper nitrate. To this solution, 315 grams of Isoleucine is slowly added. The solution is heated at about 50° C. for 8 hours. The product is cooled, and dried yielding a nitrate-complexed cupric bisisoleucine amino acid chelate.

### Example 12

### Nitrate Complexed Magnesium Histidine Chelate

To about 1200 ml of deionized water containing 71 grams nitric acid, 170 grams of histidine is added. To this solution of nitric acid and histidine, 115 grams of potassium nitrate is slowly added. The solution is heated at about 50° C. for 8 hours. The product is cooled, and dried yielding a nitrate-complexed potassium magnesium histidine amino acid chelate.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A nitrate-complexed amino acid chelate composition, comprising a metal, an amino acid ligand, and a nitrate, wherein the amino acid ligand is chelated to the metal forming an amino acid chelate and the nitrate is complexed to the amino acid chelate.

2. The composition of claim 1, wherein the amino acid ligand is selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine, including dipeptides, tripeptides, and tetrapeptides thereof.

3. The composition of claim 1, wherein the metal is selected from the group consisting of copper, zinc, manganese, iron, chromium, calcium, potassium, sodium, magnesium, cobalt, nickel, molybdenum, vanadium, strontium, selenium, silicon, and combinations thereof.

4. The composition of claim 1, wherein the nitrate-complexed amino acid chelate has an amino acid ligand to metal ratio from about 1:1 to about 3:1.

5. The composition of claim 1, wherein the nitrate-complexed amino acid chelate has a nitrate to amino acid chelate ratio from about 0.1:1 to about 1:3.

6. The composition of claim 1, wherein the nitrate-complexed amino acid chelate includes a compound comprising 2 amino acid ligands and 1 nitrate.

7. The composition of claim 1, wherein the nitrate-complexed amino acid chelate includes a compound comprising 3 amino acid ligands and 1 nitrate.

8. The composition of claim 1, wherein the nitrate-complexed amino acid chelate includes a compound comprising 1 amino acid ligand and 1 nitrate.

9. The composition of claim 1, further comprising a second amino acid chelate admixed with the nitrate-complexed amino acid chelate, said second amino acid chelate comprising a second amino acid ligand chelated to a second metal.

10. The composition of claim 9, wherein the second amino acid chelate is selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine, including dipeptides, tripeptides, and tetrapeptides thereof.

11. The composition of claim 9, wherein the second metal is selected from the group consisting of copper, zinc, manganese, iron, chromium, calcium, potassium, sodium, silicon, magnesium, cobalt, nickel, molybdenum, vanadium, strontium, and selenium.

12. The composition of claim 9, wherein the second amino acid chelate is a second nitrate-complexed amino acid chelate.

13. The composition of claim 9, wherein the second amino acid chelate is a nitrate-chelated amino acid chelate.

14. The composition of claim 9, wherein the nitrate-complexed amino acid chelate is admixed with the second amino acid chelate in a ratio of about 1:10 to about 10:1.

15. The composition of claim 1, further comprising a nitrate salt admixed with the nitrate-complexed amino acid chelate.

16. The composition of claim 15, wherein the nitrate salt is selected from the group consisting of group 1 element nitrates; group 2 element nitrates; transitional metal nitrates; amino acid nitrates; quaternary amine nitrates; mono-, di-, and trimethylaminenitrates; mixtures thereof; and derivatives thereof.

17. The composition of claim 15, wherein the nitrate-complexed amino acid chelate is admixed with the nitrate salt in a ratio of about 1:10 to about 10:1.

18. A nitrate-chelated amino acid chelate composition, comprising a metal, an amino acid ligand, and a nitrate, wherein the amino acid ligand and the nitrate are chelated to the metal forming a nitrate-chelated amino acid chelate.

19. The composition of claim 18, wherein the amino acid ligand is selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine, including dipeptides, tripeptides, and tetrapeptides thereof.

20. The composition of claim 18, wherein the metal is selected from the group consisting of copper, zinc, manganese, iron, chromium, calcium, potassium, sodium, magnesium, cobalt, nickel, molybdenum, vanadium, strontium, selenium, silicon, and combinations thereof.

21. The composition of claim 18, wherein the nitrate-chelated amino acid chelate has an amino acid ligand to metal ratio from about 1:1 to about 3:1.

22. The composition of claim 18, wherein the nitrate-chelated amino acid chelate has a nitrate to metal ratio from about 0.1:3 to about 3:1.

23. The composition of claim 18, wherein the nitrate-chelated amino acid chelate includes a compound comprising 2 amino acid ligands and 1 nitrate.

24. The composition of claim 18, wherein the nitrate-chelated amino acid chelate includes a compound comprising 3 amino acid ligands and 1 nitrate.

25. The composition of claim 18, wherein the nitrate-chelated amino acid chelate includes a compound comprising 1 amino acid ligand and 1 nitrate.

26. The composition of claim 18, wherein the nitrate-chelated amino acid chelate includes a compound comprising 2 amino acid ligands and 2 nitrates.

27. The composition of claim 18, wherein the nitrate-chelated amino acid chelate includes a compound comprising 1 amino acid ligand and 3 nitrates.

28. The composition of claim 18, wherein the nitrate-chelated amino acid chelate includes a compound comprising 1 amino acid ligand and 2 nitrates.

29. The composition of claim 18, further comprising a second amino acid chelate admixed with the nitrate-chelated amino acid chelate, said second amino acid chelate comprising a second amino acid ligand chelated to a second metal.

30. The composition of claim 29, wherein the second amino acid chelate is selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine, including dipeptides, tripeptides, and tetrapeptides thereof.

31. The composition of claim 29, wherein the second metal is selected from the group consisting of copper, zinc, manganese, iron, chromium, calcium, potassium, sodium, silicon, magnesium, cobalt, nickel, molybdenum, vanadium, strontium, and selenium.

32. The composition of claim 29, wherein the second amino acid chelate is a second nitrate-chelated amino acid chelate.

33. The composition of claim 29, wherein the second amino acid chelate is a nitrate-complexed amino acid chelate.

34. The composition of claim 29, wherein the nitrate-chelated amino acid chelate is admixed with the second amino acid chelate in a ratio of about 1:10 to about 10:1.

35. The composition of claim 18, further comprising a nitrate salt admixed with the nitrate-chelated amino acid chelate.

36. The composition of claim 34, wherein the nitrate salt is selected from the group consisting of group 1 element nitrates; group 2 element nitrates; transitional metal nitrates; amino acid nitrates; quaternary amine nitrates; mono-, di-, and trimethylaminenitrates; mixtures thereof; and derivatives thereof.

37. The composition of claim 34, wherein the nitrate-chelated amino acid chelate is admixed with the nitrate salt in a ratio of about 1:10 to about 10:1.

38. A method of increasing a metabolic activity in an animal tissue, comprising administering a nitrate amino acid chelate composition including a metal, an amino acid ligand, and a nitrate; wherein the amino acid ligand is chelated to the metal forming an amino acid chelate and the nitrate is chelated or complexed to the amino acid chelate, wherein the composition is administered to an animal in an amount sufficient to i) raise the metal concentration within the tissue, ii) retain metal content in the tissue for a greater period of time compared to when the metal is delivered as a non-nitrate-containing compound, and iii) enhance metabolic activity of the tissue.

39. The method of claim 38, wherein the amino acid ligand is selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine, including dipeptides, tripeptides, and tetrapeptides thereof.

40. The method of claim 38, wherein the metal is selected from the group consisting of copper, zinc, manganese, iron, chromium, calcium, potassium, sodium, silicon, magnesium, cobalt, nickel, molybdenum, vanadium, strontium, and selenium.

41. The method of claim 38, wherein the animal is a mammal.

42. The method of claim 38, wherein the animal is a human.

43. The method of claim 38, wherein the animal is a fowl.

44. The method of claim 38, wherein the animal is a fish.

45. The method of claim 38, wherein the animal is a crustacean.

46. The method of claim 38, wherein the metabolic activity is milk production, enhanced growth, enhanced fertility, reduced morbidity, reduced tissue fat, or enhanced feed conversion.

47. The method of claim 38, wherein the step of administering is by a formulation selected from the group consisting of oral, injection, powder, tablet, capsule, gel, liquid, or paste.

48. The method of claim 47, wherein the step of administering is by oral administration.

49. The method of claim 38, including co-administering a second amino acid chelate that is different than the amino acid chelate, said second amino acid chelate including a second metal and a second amino acid ligand.

50. The method of claim 49, wherein the second amino acid ligand is selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine, including dipeptides, tripeptides, and tetrapeptides thereof.

51. The method of claim 49, wherein the second metal is selected from the group consisting of copper, zinc, manganese, iron, chromium, calcium, potassium, sodium, silicon, magnesium, cobalt, nickel, molybdenum, vanadium, strontium, and selenium.

52. The method of claim 49, wherein the second amino acid chelate is a second nitrate-complexed amino acid chelate.

53. The method of claim 49, wherein the second amino acid chelate is a nitrate-chelated amino acid chelate.

54. The method of claim 49, wherein the metal and the second metal are the same, and the amino acid ligand and the second amino acid ligand are different.

55. The method of claim 49, wherein the metal and the second metal are different, and the amino acid ligand and the second amino acid ligand are different.

56. The method of claim 49, wherein the metal and the second metal are different, and the amino acid ligand and the second amino acid ligand are the same.

57. The method of claim 49, wherein the amino acid chelate and the second amino acid chelate each have an amino acid ligand to metal ratio from about 1:1 to about 3:1.

58. The method of claim 49, wherein the nitrate-complexed amino acid chelate composition has a nitrate-complexed amino acid chelate to second amino acid chelate ratio from about 10:1 to about 1:10.

59. The method of claim 38, further comprising a nitrate salt admixed with the nitrate-complexed amino acid chelate.

60. The method of claim 58, wherein the nitrate salt is selected from the group consisting of group 1 element nitrates; group 2 element nitrates; transitional metal nitrates; amino acid nitrates; quaternary amine nitrates; mono-, di-, and trimethylaminenitrates; mixtures thereof; and derivatives thereof.

61. The method of claim 58, wherein the nitrate-complexed amino acid chelate is admixed with the nitrate salt in a ratio of about 1:10 to about 10:1.

62. The method of claim 38, wherein the nitrate amino acid chelate is a nitrate-complexed amino acid chelate.

63. The method of claim 38, wherein the nitrate amino acid chelate is a nitrate-chelated amino acid chelate.

64. A method of increasing a metabolic activity in a plant, comprising administering a nitrate amino acid chelate composition including a metal, an amino acid ligand, and a nitrate; wherein the amino acid ligand is chelated to the metal metal forming an amino acid chelate and the nitrate is chelated or complexed to the amino acid chelate, wherein the composition is administered to the plant in an amount sufficient to raise the nitrogen concentration within the plant and enhance metabolic activity of the plant.

65. The method of claim 64, wherein the amino acid ligand is selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine, including dipeptides, tripeptides, and tetrapeptides thereof.

66. The method of claim 64, wherein the metal is selected from the group consisting of copper, zinc, manganese, iron, chromium, calcium, potassium, sodium, silicon, magnesium, cobalt, nickel, molybdenum, vanadium, strontium, and selenium.

67. The method of claim 64, wherein the nitrate salt is selected from the group consisting of group 1 element nitrates; group 2 element nitrates; transitional metal nitrates; amino acid nitrates; quaternary amine nitrates; mono-, di-, and trimethylaminenitrates; mixtures thereof; and derivatives thereof.

68. The method of claim 64, wherein the metabolic activity is enhanced growth, enhanced fruit production, reduced morbidity, or enhanced fruit size.

69. The method of claim 64, wherein the step of administering is by a formulation selected from the group consisting of foliar plant fertilizer, solid plant fertilizer, liquid plant fertilizer, or combinations thereof.
